(19) United States
(12) Patent Application Publication
(10) Pub. No.: US 2012/0143100 A1
(43) Pub. Date: Jun. 7, 2012

(54) EXTENDED DEPTH-OF-FOCUS HIGH INTENSITY ULTRASONIC TRANSDUCER

(75) Inventors: Jong Seob Jeong, Los Angles, CA (US); K. Kirk Shung, Los Angeles, CA (US)

(73) Assignee: UNIVERSITY OF SOUTHERN CALIFORNIA, Los Angeles, CA (US)

(21) Appl. No.: 13/390,496

(22) PCT Filed: Aug. 16, 2010
(86) PCT No.: PCT/US10/45641

§ 371 Date: Feb. 14, 2012

Related U.S. Application Data
(60) Provisional application No. 61/234,171, filed on Aug. 14, 2009.

(51) Int. Cl. A61N 7/00

(52) U.S. Cl. 601/2

(57) ABSTRACT

Compound ultrasonic transducers are disclosed that include multiple transducer elements configured to have overlapping or consecutive depths of focus in an axial direction. Embodiments of such transducers can be composed of a concentric disc- and one or more annular-type elements and each element has a different radius of curvature to produce multiple focal zones (depths-of focus) upon excitation. Exemplary embodiments include a concentric disc- and one annular-type elements, with each element has a different radius of curvature, which are referred to herein as a dual-focus therapeutic ultrasound transducer (DFTUT). Method of directing ultrasonic energy to a target area and thereby aligning ultrasonic focal zones (depths-of focus) are also disclosed. Two or more transducer elements can each be configured to produce a different focal zone at the target area, such as targeted tissue.

### CROSS-REFERENCE TO RELATED APPLICATIONS

This application is a national phase filing under 35 U.S.C 371 of PCT Application No. PCT/US2010/045641, filed Aug. 16, 2010, entitled “Extended Depth-of-Focus High Intensity Ultrasonic Transducer,” attorney docket no. 028080-0593, which claims the benefit of U.S. Provisional Patent Application No. 61/234,171 entitled “Method to Extend Depth-of-Field of High Intensity Focused Ultrasound Transducer,” filed 14 Aug. 2009, attorney docket no. 028080-0499. The entire content of both applications is incorporated herein by reference.

### BACKGROUND

In recent years, high intensity focused ultrasound (“HIFU”) has become increasingly important in the noninvasive treatment of malignant tissues. Several clinical studies have been conducted to investigate the feasibility of HIFU treatment for breast, liver, and prostate cancer. HIFU therapy is usually performed in cooperation with medical imaging modalities such as magnetic resonance imaging (“MRI”), ultrasound imaging, and computed tomography (“CT”) in order to select and monitor a treatment region. MRI provides a high-resolution image and an efficacious temperature map, but it is expensive and requires a large space. Ultrasound is another common tool for image guidance. It offers advantages in real-time imaging, cost-effectiveness, excellent portability, and potential integration with other devices.

Several techniques have been developed to decrease the treatment time of noninvasive ultrasound therapy. One of them is the generation of multi-foci with a phased array transducer capable of electronic focusing and steering (Daum et al. 1999; Ebbini et al. 1989; Wan H et al. 1996). Also, multi-zone transmit focusing which is usually used for imaging can increase ablated tissue lesions in the axial direction (Do-Huu and Hartemann 1982). However, the conventional multi-foci technique and multi-zone transmit focusing scheme requires a complex array architecture. Also, numerous electronic components including high-power amplifiers and time delay components are required to activate and control each element independently. Although several investigators have used specially designed lenses and conical shape transducers to increase depth-of-focus (DOF), most studies were focused on enhancing imaging performance (Burckhardt et al. 1973; Patterson and Foster 1982; Trzaskos and Young 1987). A few researchers have proposed a split-focusing technique to generate multi-foci simultaneously with a geometrically divided transducer, or a transducer with sectional electrode (Patel et al. 2008; Sasaki et al. 2003; Seip et al. 2001) driven by voltages of different phases. Recently, the toric transducer was developed to generate large ablated lesions (Melodelima et al. 2009). The aforementioned studies have focused on producing broad tissue lesions in the lateral and elevational directions.

What is desired therefore are improved ultrasonic techniques for treatment of tissues.

### SUMMARY

The present disclosure in general terms is directed to novel apparatus and methods utilizing a compound ultrasonic transducer providing an extended depth-of-focus (DOF).

An aspect of the present disclosure is directed to a multi-element ultrasonic transducer that includes two or more transducer elements that are each configured to produce a different focal zone at a target area, such as along an axis in desired treatment area of tissue. Examples of the multi-element transducer can include a disc-type element surrounded by two or more annular-type elements of different radii of curvatures to produce multiple focal zones. To increase focal depth and to maintain uniform beamwidth of the elongated DOF, each element can transmit ultrasound of a different center frequency. For example, an inner element can operate at a higher frequency for near field focusing and an outer element can operate at a lower frequency for far field focusing. By activating the multiple elements at the same time, e.g., with a single transmitter capable of generating a multiple-frequency mixed signal, an extended DOF can be produced.

An exemplary embodiment of a transducer according to the present disclosure includes a disc-type and an annular-type element of different radii of curvatures, a 4.1 MHz inner element and a 2.7 MHz outer element, to produce two focal zones.

A further aspect of the present disclosure is directed to a method of directing ultrasonic energy to a target area and thereby aligning ultrasonic focal zones (depths-of field), such as along an axis in desired treatment area of tissue. Two or more transducer elements can each be configured to produce a different focal zone at the target area. Activating the multiple elements at the same time, e.g., with a single transmitter capable of generating a multiple-frequency mixed signal, can produce an extended overall DOF of the transducer at the target area.

An exemplary embodiment of a method of directing ultrasonic energy to a target area can include using the HFUS output of a multi-element transducer for effecting thrombolysis in targeted tissue. The method can include providing a multi-element ultrasonic transducer including a disc-type element surrounded by an annular-type element of different radii of curvatures to produce multiple focal zones. The transducer can include a disc-type and an annular-type element of different radii of curvatures, a 4.1 MHz inner element and a 2.7 MHz outer element, to produce two focal zones. Activating the multiple elements at the same time can include using a single transmitter capable of generating a multiple-frequency mixed signal. Directing the HFUS output to the targeted tissue can accordingly produce thrombolysis in the targeted tissue.

While aspects of the present disclosure are described herein in connection with certain embodiments, it is noted that variations can be made by one with skill in the applicable arts within the spirit of the present disclosure and the scope of the appended claims.

### BRIEF DESCRIPTION OF THE DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 depicts a schematic diagram, including (a) side view and (b) front view, of a dual-focus therapeutic ultrasound transducer (DFTUT) for a system/method, in accordance with exemplary embodiments of the present disclosure;

FIG. 2 depicts a set of schematic diagrams of a DFTUT aperture used in a Field-II simulation, in accordance with exemplary embodiments of the present disclosure;

FIG. 3 depicts a set of transmit beam profiles of the inner element with 19 mm focal depth: (a) a contour plot in the decibel scale, (b) lateral beam profile, and (c) axial beam profile, in accordance with exemplary embodiments of the present disclosure;

FIG. 4 depicts a set of transmit beam profiles of the outer element with 24 mm focal depth: (a) a contour plot in the decibel scale, (b) lateral beam profile, and (c) axial beam profile, in accordance with an embodiment of the present disclosure;

FIG. 5 depicts a set of transmit beam profiles of the DFTUT: (a) a contour plot in the decibel and (b) axial beam profile, in accordance with an embodiment of the present disclosure;

FIG. 6 depicts a set of transmit beam profiles of the single focused transducer with 21.5 mm focal depth: (a) a contour plot in decibel, (b) lateral beam profile, and (c) axial beam profile, according to an exemplary embodiment of the present disclosure;

FIG. 7 FIG. depicts a photograph of the prototype DFTUT., in accordance with exemplary embodiments of the present disclosure;

FIG. 8 depicts a measured electrical impedance of the DFTUT with a water load, in accordance with an exemplary embodiment of the present disclosure;

FIG. 9 depicts a schematic diagram of an experimental setup for measurement of the transmit response, DOF, and lateral beamwidth of the DFTUT by using a hydrophone, in accordance with exemplary embodiments of the present disclosure;

FIG. 10 depicts a set of frequency domain plots of the measured transmit response along the axial direction: (a) 18 mm, (b) 23 mm, (c) 28 mm, and (d) 33 mm in depth, in accordance with exemplary embodiments of the present disclosure;

FIG. 11. depicts a set of plots with simulated and measured data for the DFTUT using a hydrophone: (a) an axial beam profile with DOF and (b)-6 dB overall lateral beamwidth within the −6 dB DOF, in accordance with an exemplary embodiment of the present disclosure;

FIG. 12 depicts a plot with simulated and measured lateral beam pattern for the DFTUT: the simulated and the measured data at 20 mm and at 22 mm in depth (blue-dashed)., in accordance with an embodiment of the present disclosure;

FIG. 13 depicts a set of plots of (a) Simulated temperature distribution and (b) thermal dose for the tested DFTUT by targeting a liver layer; and

FIG. 14 depicts a schematic view of a DFTUT and a cross-section of a piece of beef liver after HIFU sonication with DFTUT. The arrow indicates the HIFU exposure direction. (a) A schematic diagram to generate (b) the ablated lesion in a beef liver.

While certain embodiments depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

In general terms, aspects of the present disclosure are directed to novel apparatus and methods utilizing a compound ultrasonic transducer providing an extended depth-of-focus. Such techniques can provide a reduction in the treatment time of large tumors in high intensity ultrasound therapy by increasing DOF in the axial direction.

Compound ultrasonic transducers according to the present disclosure include multiple transducer elements configured to have overlapping or consecutive depths of focus in an axial direction. Embodiments of such transducers can be composed of a concentric disc- and one or more annular-type elements and each element has a different radius of curvature to produce multiple focal zones upon one excitation. As described in further detail below, exemplary embodiments include a multi-element transducer including a concentric disc-type element and an annular-type element, with each element has a different radius of curvature, which are referred to herein as a dual-focus therapeutic ultrasound transducer (DFTUT).

Different center frequencies can be used for simultaneous multi-zone transmit focusing and thus result in an enhanced focal depth. Each element can be made of a piezoelectric composite material of a different thickness and subsequently optimized for transmitting ultrasound corresponding to its own resonant frequency. Thus, the multiple elements can work like band-pass filters of the excitation signal. These properties enable the transducers to be activated by a single transmitter capable of generating a multi-frequency mixed signal. The center frequency and the dimension of each element can be optimized based on the application considering a target size and distance. Also, the relative geometric focus offset between two different focal points and the output power of each element may affect the uniformity of the extended DOF and the lateral beamwidth. A good alignment between these two elements in the fabrication process is desired to achieve a uniform compound beam profile in the axial and lateral direction.

Clinically the efficacy of a focused therapeutic transducer may be estimated from its −6 dB intensity contour of the focal zone. Along the axis of propagation, the effective focal zone is defined by the DOF which is related to the square of the f-number (focal depth/aperture size) and the wavelength.

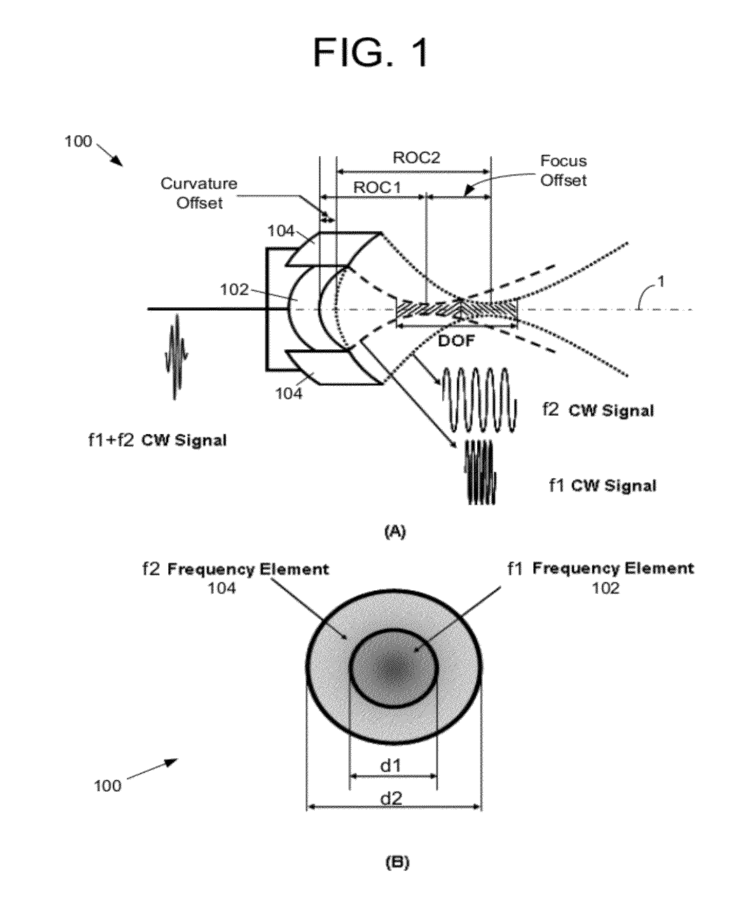

FIG. 1 depicts a schematic diagram including two views, (a) side view and (b) front view, of a dual-focus therapeutic ultrasound transducer (DFTUT) used for a system/method 100, in accordance with exemplary embodiments of the present disclosure. The DFTUT system/method 100 can provide an increase in the DOF while maintaining the necessary ultrasound intensity level for therapy.

The DFTUT system/method 100 can utilize a disc-type-inner element 102 and an annular-type-outer element 104 with different radii of curvatures (shown by ROC1 and ROC2) and different diameters, d1 and d2. Each element (102 and 104) can be made of a piezoelectric composite material of a different thickness and subsequently be optimized for transmitting ultrasound corresponding to its own resonant frequency (shown by f1 and f2, respectively). The respective DOF of each element can be aligned along an axis 1 (in an axial direction), as indicated. Of course, the number of annular elements can be increased (e.g., in a nested configuration with different diameters, respectively) depending on the desired overall DOF. The overall DOF for the multi-element transducer can be adjusted by adjusting the degree of overlap of the respective DOF regions generated by the respective elements. For example, the overall DOF can be increased by reducing or minimizing the overlap of the respective DOF regions. Conversely, the overall DOF can be decreased by increasing the overlap of the respective DOF regions.

With continued reference to FIG. 1, the electrodes of the these elements, 102 and 104, can be connected together allowing ultrasound at two different frequencies to be simultaneously emitted to the two focal zones. For driving the transducer elements, a single transmitter that generates a continuous wave (CW) signal at the multiple frequencies, e.g., two, f1 and f2, can be used. Alternatively, multiple transmitters can be used, e.g., one for each different frequency of the respective multiple different transducer elements; for such applications, each transducer can driven by a separate transmitter with relatively low power consumption compared to a single transmitter that drives all of the transducer elements. The relative intensity for each element can be controlled by changing the amplitude of the two frequency components in the input signal. The electrical impedance for inner and outer elements are preferably matched to that of the driver and/or controller system (not shown) mainly with a power amplifier for balanced output power.

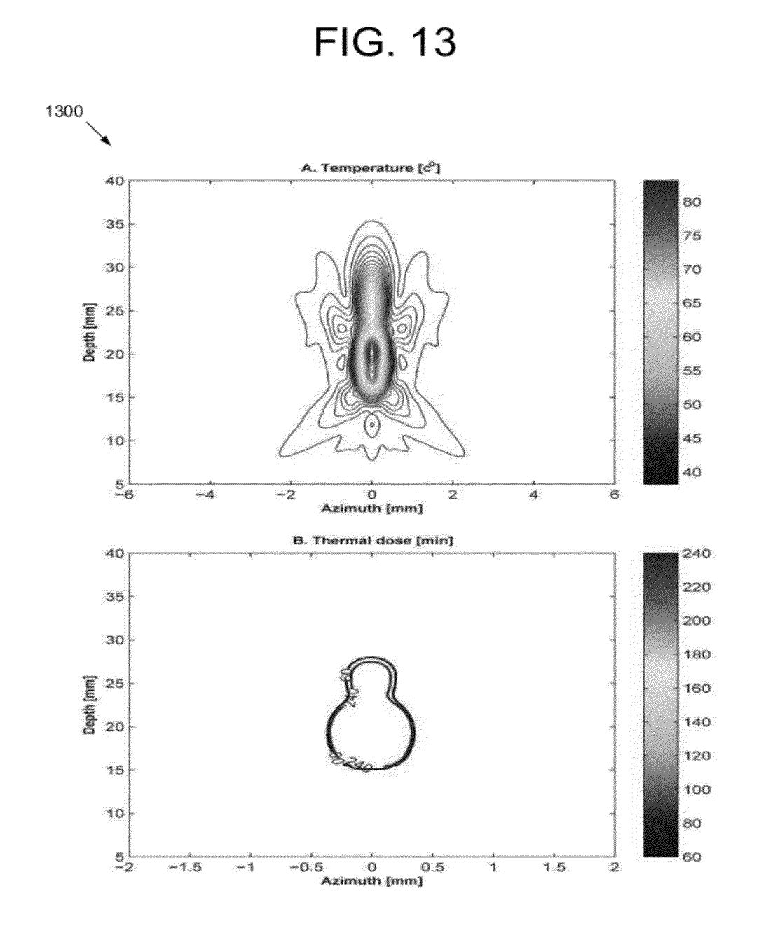

To experimentally verify efficacy, a prototype DFTUT was fabricated and its performance was demonstrated by hydrophone measurements and lesion formation tested on a piece of beef liver, as described in further detail for FIG. 2-14. Also, as shown in FIG. 13, numerical simulations for the thermal effect on a tissue with bio-heat transfer/thermal dose equations yielded results on the estimated temperature/thermal dose distribution in the extended DOF for the DFTUT.

The prototype DFTUT, used for simulation and the preliminary experiments, included a two-element transducer having a single annular element concentric with an inner circular element. The inner and outer elements of the DFTUT had 4.1 MHz and 2.7 MHz center frequencies with 19 mm and 24 mm radii of curvatures, respectively. In this case the relative geometric focus offset between two focal depths (shown in FIG. 1) was 5.24 mm. The center frequencies, dimensions, and radii of curvatures are not limited to such and can of course be chosen based on applications. The relative geometric focus offset was determined considering the overlapped zone of two elements at higher than −6 dB. The electrodes of the these elements were connected together allowing ultrasound at two different frequencies to be simultaneously emitted to the two focal zones with a single transmitter that was operable to generate a continuous wave (CW) signal at two-frequencies. The relative intensity for each element was controlled by changing the amplitude of the two frequency components in the input signal. The electrical impedance for inner and outer elements was matched to that of the system mainly with a power amplifier for balanced output power. The diameters of the inner and outer elements were 12 mm and 21 mm, respectively. Under these conditions, the −6 dB lateral beamwidth of the DFTUT was similar to those obtained by inner and outer elements excited independently.

The prototype DFTUT was built following the specifications summarized in Table 3. The transducer elements were made of 1-3 piezoelectric composite elements using PZT4 (840, APC Company, Mackeyville, Pa.) and epoxy (EPO-TEK314, Epoxy Technology, Billerica, Mass.), which facilitated spherically shaping and reduction of acoustic impedance mismatch between the transducers and the tested acoustic medium. PZT4 has high Curie temperature and high mechanical Q and thus is an excellent material for therapeutic transducer designs. Also, the epoxy used has a high glass transition temperature (100° C.) which makes it less susceptible to failure during operation. For the 4.1 MHz inner element, a pre-poled PZT4 plate was diced with a 35 μm wide blade to make the 1-3 composite. The composite pitch was 250 μm and the post-widths were 215 μm. After a cleaning and drying procedure, the kerfs were filled with unloaded EPO-TEK 314. After the composite was cured at 120° C. for 3 hours, the composite was then lapped to a final thickness of 450 μm and heat pressed to a spherically curved shape at 130° C. using a rubber mold and a chrome/steel ball. The same process was used to fabricate the outer ring element. After heat pressing, the two elements were carefully bonded together, and a 0.5 μm thick gold/chrome-sputtered layer was used as the electrode for both the signal and ground electrodes. No matching and backing layers were used in the prototype transducer in order to minimize heat absorption and to maximize transmit intensity. FIG. 7 shows a photograph of the finished prototype DFTUT. The measured TAP (Total Acoustic Power) was 11 W, the Iₛₚₜₐ (spatial-peak temporal-average intensity) was 1412 W/cm² and the energy conversion efficiency was 53%. Note that all data were measured by driving two elements simultaneously.

FIG. 2 depicts a set 200 of schematic diagrams including two perspective views, A-B, of a DFTUT aperture used in a Field-II simulation, in accordance with exemplary embodiments of the present disclosure. As shown in FIG. 2, the on-axis transmit beam profile was computed with the Field-II (Jensen and Svendaenl 1991) simulation program derived from the Tupholme-Stepanishen method (Stepanishen 1971; Tupholme 1969). The two signals were simultaneously emitted to the target via the inner and outer elements of the dual curved aperture in order to model the amplitude and phase interaction between two waves of different frequencies in the transmit-field. The dual curved aperture was created with Field-II's automatic meshing aperture generation as shown in FIG. 2, and two waveforms of different frequencies were assigned to each element. This method was compared to post-sum approach, i.e., sum of two transmit-field profiles for each aperture. There are low level discrepancies in the time and frequency responses of scanlines and final beam profiles implemented by these two schemes given that the simulation is an approximation. In the final beam profiles, the difference between two schemes in −6 dB and −20 dB axial beamwidths were about 1 mm and 2 mm, respectively.

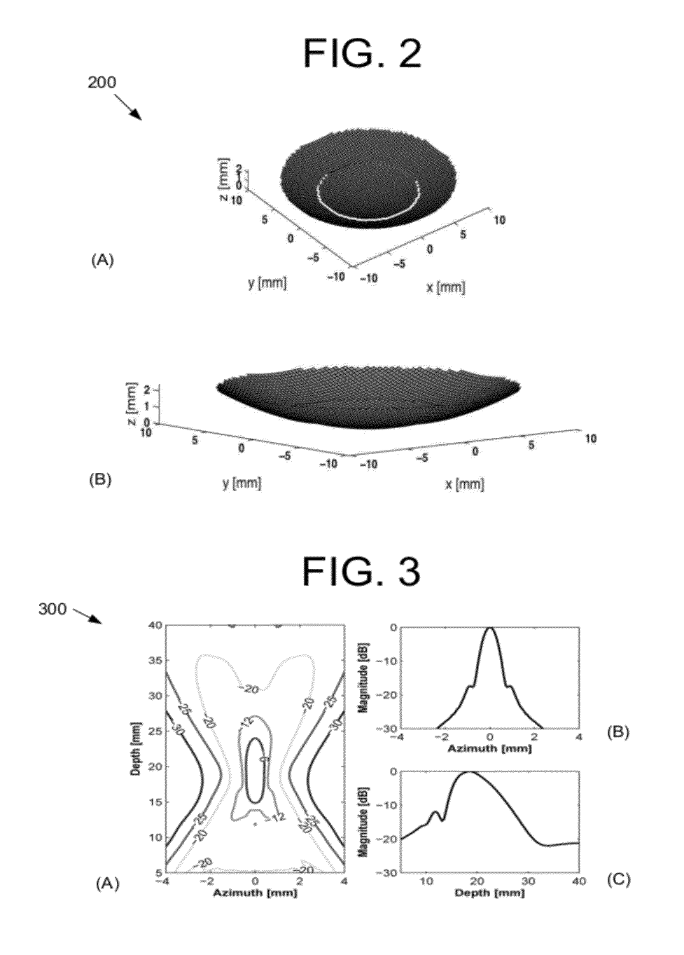

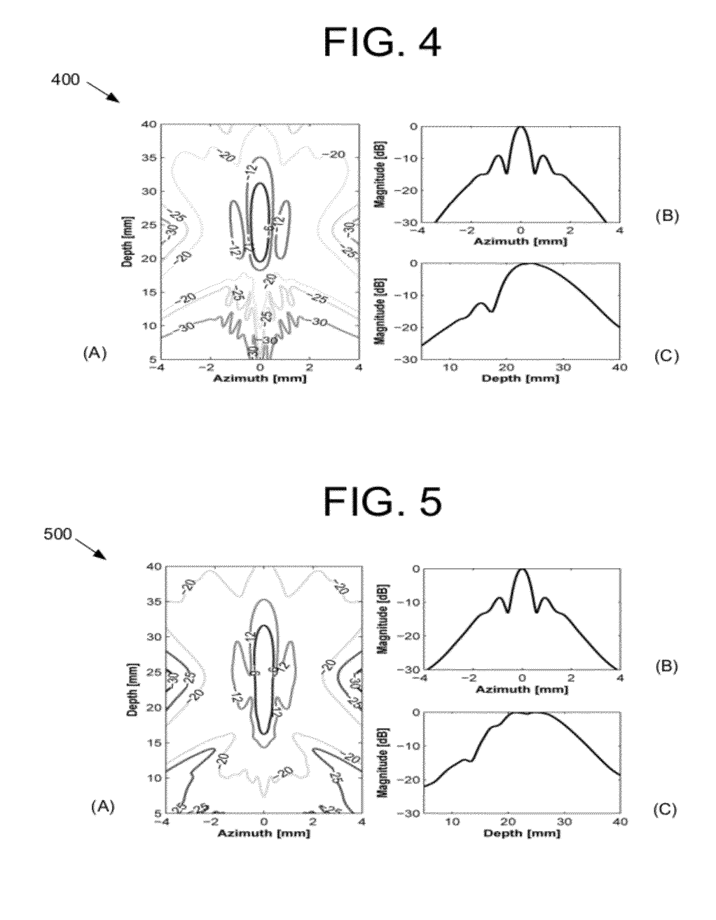

The transducer aperture was apodized using the Hanning window and segmented by 300 μm rectangular elements. The radii of curvatures for inner and outer elements are 19 mm and 24 mm, respectively. The 4.1 MHz and 2.7 MHz input signals of 30%-6 dB bandwidth were assigned to each aperture based on the expected output from the prototype transducer without a backing layer. The sampling frequency was 40 times the 4.1 MHz frequency, and the amplitude of each frequency component was the same. The lateral and axial grid sizes were 20 μm and 100 μm, respectively. The relative geometric focus offset between two focal points was 5.24 mm. At this distance, the −2 dB contours off the maximal peaks for the two signals in FIG. 3 and FIG. 4 were shown to cross each other.

Table 1 shows the specification of the DFTUT. Two kinds of simulations were conducted using the properties of the two different media such as water and liver. The results of these simulations are summarized in Table 2 and are displayed for water only in FIGS. 3-5.

TABLE 1
Specifications of the DFTUT for the sound field simulation

| | Inner Element | | Outer Element | |
|---|---|---|---|---|
| Center frequency | 4.1 | MHz | 2.7 | MHz |
| Diameter | 12 | mm | 21 | mm |
| Geometric focus | 19 | mm | 24 | mm |
| F-number | 1.6 | | 1.1 | |
| Relative geometric focus offset | 0 | mm | 5.64 | mm |
| Water attenuation coefficient^{a} | 0.002 | dB/cm MHz² | | |
| Liver attenuation coefficient^{b} | 0.43 | dB/cm MHz | | |

^{a}(Shung 2006)

TABLE 2
Simulated −6 dB DOF, −6 dB lateral beamwidth,
and sidelobe for several transducers

Medium

| | | Water | Liver |
|---|---|---|---|
| 4.1 MHz Inner | −6 dB DOF [mm] | 9.1 | 9.2 |
| Element | −6 dB Lateral | 0.81 | 0.85 |
| | beamwidth [mm] | | |
| | Sidelobe [dB] | −17 | −17.55 |
| 2.7 MHz Outer | −6 dB DOF [mm] | 11.7 | 11.9 |
| Element | −6 dB Lateral | 0.72 | 0.76 |
| | beamwidth [mm] | | |
| | Sidelobe [dB] | −9 | −9.2 |
| 3.4 MHz Single | −6 dB DOF [mm] | 4.4 | 4.7 |
| Element | −6 dB Lateral | 0.64 | 0.68 |
| | beamwidth [mm] | | |
| | Sidelobe [dB] | −18 | −17.3 |
| DFTUT | −6 dB DOF [mm] | 15.4 | 14.7 |
| | −6 dB Lateral | 0.75 | 0.73 |
| | beamwidth [mm] | | |
| | Sidelobe [dB] | −9 | −9.4 |

FIG. 3 depicts a set 300 of transmit beam profiles of the inner element with 19 mm focal depth, as used for the prototype transducer: (a) a contour plot in the decibel scale, (b) lateral beam profile, and (c) axial beam profile, in accordance with exemplary embodiments of the present disclosure;

FIG. 4 depicts a set 400 of transmit beam profiles of the outer element with 24 mm focal depth, as used for the prototype transducer: (a) a contour plot in the decibel scale, (b) lateral beam profile, and (c) axial beam profile, in accordance with an embodiment of the present disclosure;

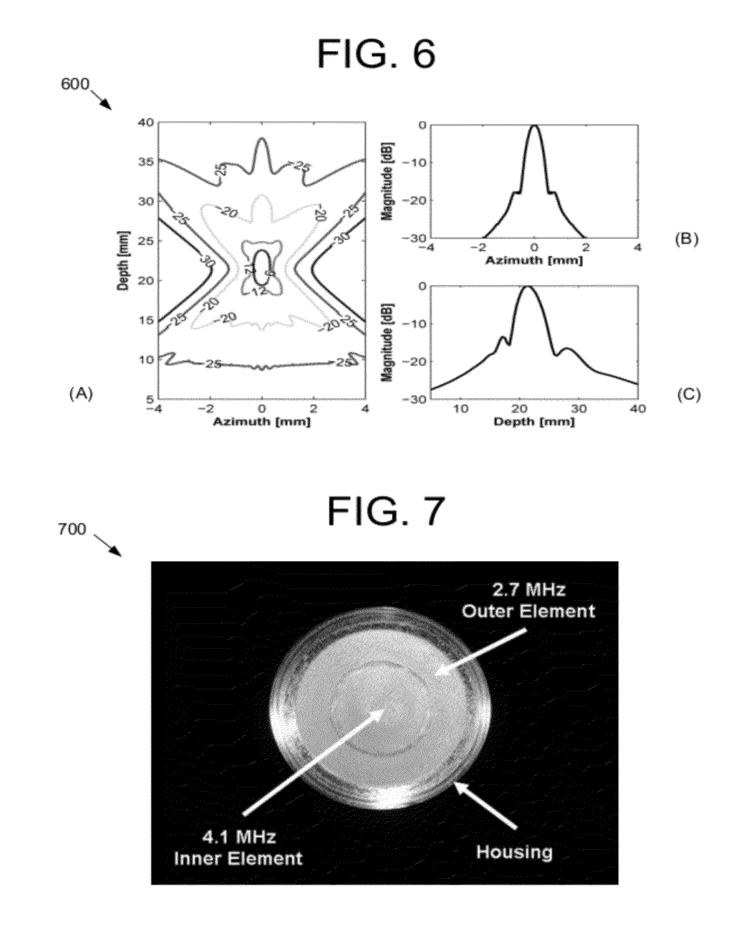

FIGS. 3-4 show the transmit beam profiles of the DFTUT's inner and outer elements excited independently. The contour plots in FIG. 3(a) and FIG. 4(a) display the relative intensity in the decibel scale. FIG. 3(b) and FIG. 4(b) show the lateral beam profile of each element at maximal peak. The −6 dB lateral beamwidths of inner and outer elements were 0.81 mm and 0.72 mm, but the sidelobes were −17 dB and −9 dB. Because outer element has a ring type aperture, the sidelobe is higher and the lateral beamwidth is narrower than a disk type aperture. FIG. 3(c) and FIG. 4(c) show the axial beam profile of each element, and −6 dB DOF were 9.1 mm and 11.7 mm, respectively. FIG. 5 shows transmit beam profile of the DFTUT at water medium. Its −6 dB DOF was 1.7 and 1.3 times larger than those obtained with inner and outer elements, respectively. The −6 dB lateral beamwidth of the proposed transducer was 0.75 mm which was close to those of inner and outer elements excited independently. The −9 dB sidelobe level was close to outer element. As a comparison, a single focused transducer with design parameters equal to the mean of the design parameters for the DFTUT (center frequency 3.4 MHz, focal depth=21.5 mm and diameter=21 mm) was also simulated (FIG. 6). The DFTUT's −6 dB DOF was 3.5 times larger, its −6 dB lateral beamwidth was 0.11 mm broader, and the sidelobe was 9 dB higher than those obtained with the single focused transducer. In the case of liver, the extended −6 dB DOF for DFTUT was 3.1 times larger than 3.4 MHz single element transducer. The sidelobe of DFTUT can be changed by controlling the dimension of outer element. Note that the medium for this simulation was single layer which was homogeneous with a single value for the attenuation coefficient.

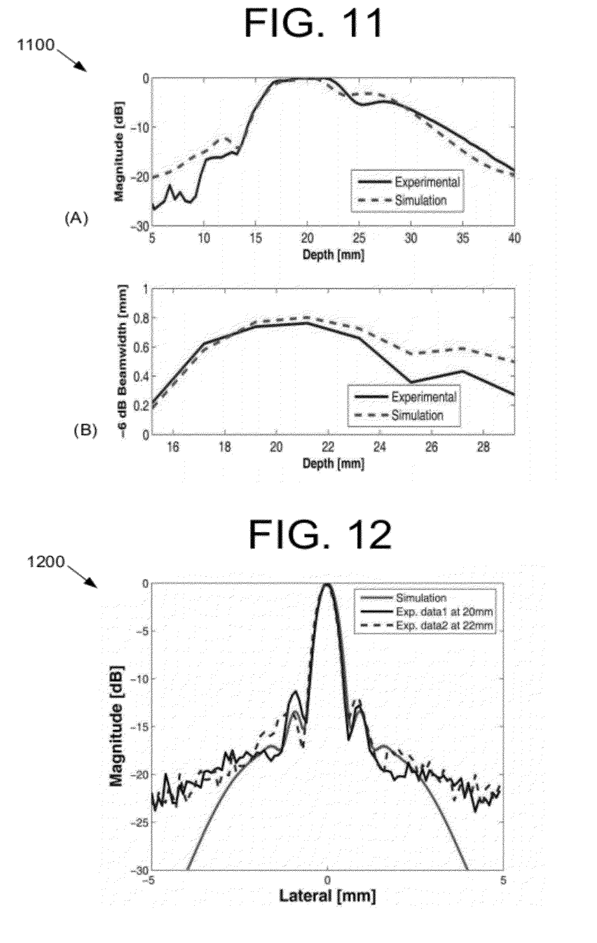

A bio-heat transfer simulation was performed with the simulated acoustic pressure field in which the amplitude for outer element was lower than inner element to obtain more realistic simulation results as shown in FIG. 11. The Pennes bio-heat transfer equation (Pennes 1948) was approximated by the equation below and numerically solved with Matlab software (The MathWorks Inc., Natick, Mass.):

$${ρ}_{t} {c}_{t} \frac{\partial T}{\partial t} = {k}_{t} {∇}^{2} T + {W}_{b} {c}_{b} ( {T}_{a} - T ) + q \qquad ( EQ . 1 )$$

Where ρₜ is the tissue density, cₜ is the specific heat of tissue, kₜ is the tissue thermal conductivity, W_{b} is the blood perfusion rate, c_{b} is the specific heat of blood, Tₐ is the arterial temperature, T is the tissue temperature, and q is the absorbed ultrasound power density defined below (Nyborg, 1981). Table 3 shows the parameters used for this simulation (Damianou et al. 1994; Damianou et al. 1997; Diederich and Burdette 1996; Fjield et al. 1996) targeting a soft tissue. Table 4 shows parameters used for bio-heat transfer simulation.

$$q = α \frac{{P}^{2}}{ρ c} \qquad ( EQ . 2 )$$

TABLE 3
Parameters used for this simulation

| | Inner Element | | Outer Element | |
|---|---|---|---|---|
| Piezoelectric material | PZT4 | | PZT4 | |
| Epoxy | EPO-TEK 314 | | EPO-TEK 314 | |
| Center frequency | 4.1 | MHz | 2.17 | MHz |
| Inner diameter | — | | 12 | mm |
| Outer diameter | 12 | mm | 21 | mm |
| Composite pitch | 250 | μm | 250 | μm |
| Composite kerf | 35 | μm | 35 | μm |
| Thickness | 450 | μm | 600 | μm |
| Volume fractional ratio | 74% | | 74% | |
| Post aspect ratio (width/thickness) | 0.48 | | 0.36 | |
| Focal depth | 19 | mm | 24 | mm |

TABLE 4
Parameters used for bio-heat transfer simulation

| | |
|---|---|
| Density (ρₜ) (kg/m³) | 1070 |
| Specific heat for tissue cₜ (J/kg/° C.) | 3770 |
| Specific heat for blood c_{b} (J/kg/° C.) | 3770 |
| Blood perfusion W_{b} (kg/m³/s) | 5 |
| Thermal conductivity kₜ (W/m/° C.) | 0.5 |
| Ultrasound absorption coefficient | 5 |
| α (Np/m/MHZ) | |

Where α is the acoustic absorption coefficient and p is the measured pressure at focal point, ρ is the density and c is the velocity. In this work, the acoustic intensity profile for the input for the equation (1) was calculated from the Field-II program using the measured acoustic pressure, 6.1 MPa. A numerical finite-difference method was used for solving the bio-heat transfer equation by replacing the derivative equation with difference quotients. The X axis range was from −4 mm to 4 mm and the Z axis range was from 1.5 mm to 46.5 mm. The X- and Z-step sizes were 0.02 mm and 0.4 mm, respectively. The time step was 0.05 seconds and simulation time was 30 seconds. FIG. 13 shows the estimated temperature distribution for DFTUT in 3D modeling. The maximum temperature recorded was 83° C. at 20 mm which is near the geometrical focus of the inner element, and the second peak has 59° C. at 26 mm which is near the geometrical focus of the outer element.

It was previously demonstrated that the thermal dose equation (EQ. 3) could predict the coagulation necrosis of the tissue based on temperature and duration of heating (Sapareto and Dewey, 1984). In this simulation, the time calculated by numerical integration at some reference temperature was equivalent to the thermal dose. Note that the temperature distribution of the bio-heat transfer equation was used for the input of the thermal dose equation (EQ. 3) (Damianou et al. 1994; Owen et al, 2010):

$${t}_{43} = {\sum }_{t = 1}^{t = final } {R}^{( 43 - {T}_{t} ) Δ t} \qquad ( EQ . 3 )$$

where, t₄₃ is the equivalent time at 43° C., and Tₜ is the average temperature during Δt. The value of R was 0.25 for temperatures lower than 43° C. and 0.5 for higher than 43° C. The lesion size was predicted with the threshold thermal dose for necrosis equivalent exposure of 43° C. for 240 min (Damianou et al. 1994).

FIG. 5 depicts a set 500 of plots of a transmit beam profile of the DFTUT: (a) a contour plot in the decibel and (b) axial beam profile, in accordance with an embodiment of the present disclosure. FIG. 5 shows the contour plot of the equivalent thermal dose distribution for 60 min and 240 min at reference 43° C. The size difference between 60 min and 240 min was lower than 0.6 mm in the axial direction. In the 240 min contour plot, the maximal lateral width and axial length were about 1 mm and 12 mm, respectively.

FIG. 6 depicts a set 600 of plots of a transmit beam profile of the single focused transducer with 21.5 mm focal depth: (a) a contour plot in decibel, (b) lateral beam profile, and (c) axial beam profile, according to an exemplary embodiment of the present disclosure.

FIG. 7 FIG. depicts a photograph 700 of the prototype DFTUT, in accordance with an exemplary embodiment of the present disclosure.

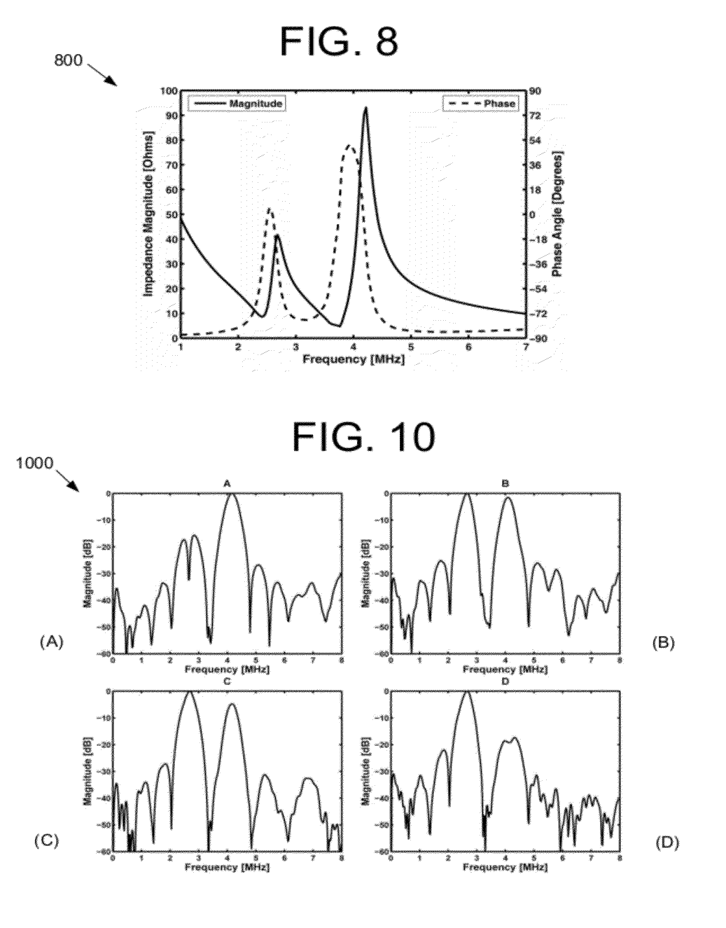

FIG. 8 depicts a plot 800 of measured electrical impedance of the DFTUT with a water load, in accordance with an exemplary embodiment of the present disclosure. FIG. 8 shows the measured electrical impedance of the water-loaded DFTUT with an impedance analyzer (4294A Impedance Analyzer, Agilent, Santa Clara, Calif.). Two peaks of the impedance plots are seen in series. One is for the outer element at a lower frequency and the other for the inner element at a higher frequency. The anti-resonance frequencies for each element were 42Ω at 2.7 MHz and 93Ω at 4.2 MHz, respectively. The driving frequencies of DFTUT were determined given a maximal peak of the transmit response resulting in a peak pressure value in the hydrophone measurement. The 2.7 MHz and 4.1 MHz frequencies for outer and inner elements yielded the highest pressures, and their impedances were 40Ω and 60Ω, respectively.

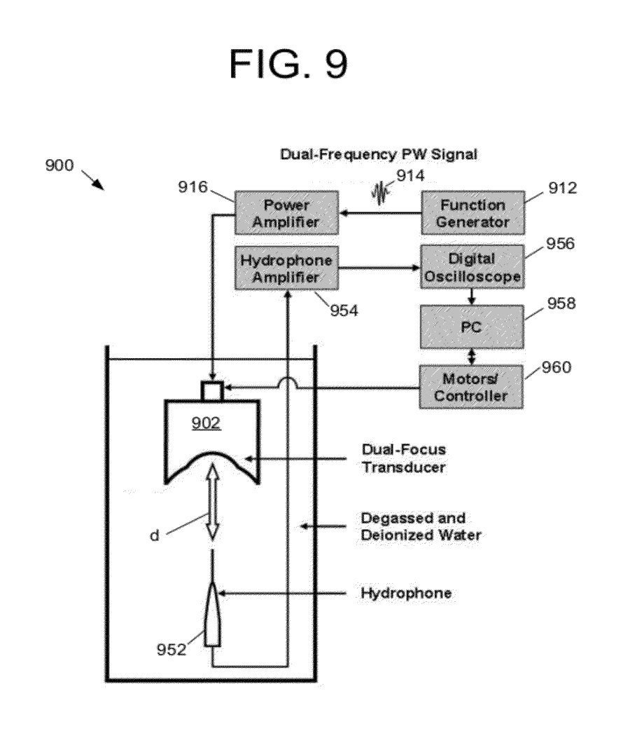

FIG. 9 depicts a schematic diagram of an experimental setup 900 for measurement of the transmit response, DOF, and lateral beamwidth of the DFTUT by using a hydrophone, in accordance with exemplary embodiments of the present disclosure. A needle hydrophone 952 (HPM04/1, Precision Acoustics Ltd, Dorchester, UK) was used to measure the transmit response of the DFTUT 902 as shown in FIG. 9. A function generator 912 (33250A, Agilent, Santa Clara, Calif.) capable of generating a 2-cycle PW signal at frequencies at 4.1 MHz and 2.7 MHz was connected to a 50 dB RF power amplifier 916 (325LA, ENI Co., Santa Clara, Calif.) resulting in 32 Vₚₚ input voltage and subsequently used to activate the DFTUT 902. The DFTUT 902 and hydrophone 952 were positioned in a container of degassed and deionized water. The distance “d” between the DFTUT 902 and the hydrophone was varied from 5 mm to 40 mm via a controller 960 (6000ULN, Burleigh Instruments Inc., Fishers, N.Y.) with a XYZ translation stage driven by a piezoelectric stepper motor 960 (IW-700 Series Inchworm Motor, Burleigh Instruments Inc., Fishers, N.Y.). Signals received by the hydrophone 952 were amplified by 25 dB (Hydrophone Booster Amplifier, Precision acoustics LTD., UK), measured with a digital oscilloscope 956 (LC534, LeCroy, Chestnut Ridge, N.Y.) with 8-bit ADC (Analog to Digital Converter) card, and recorded by a computer with a data acquisition board. A personal computer (PC) 958 and hydrophone amplifier 954 were also utilized.

FIG. 10 shows a set 1000 of plots of the measured transmit frequency domain response for the DFTUT at various depths along the axial direction. Its magnitude at different depths was observed to be proportional to the amount of energy contributed by the two elements. In the near field as shown in FIG. 10(a), 4.1 MHz frequency component of the inner element was higher than 2.7 MHz frequency, and this ratio was reversed at far field as shown in FIG. 10(b),(c),(d). The distances between a hydrophone and the transducer in FIG. 10 (a)-(d) were 18 mm, 23 mm, 28 mm, and 33 mm, respectively.

The identical experimental setup as described in FIG. 9 was used to measure the −6 dB DOF and the −6 dB lateral beamwidth of the DFTUT. The swept range for the hydrophone was from 5 mm to 40 mm in the axial direction and from −5 mm to 5 mm in the lateral direction. Eight sets of lateral beam profiles were recorded at 2 mm intervals within the −6 dB DOF, i.e., 15 mm-30 mm.

FIG. 11. depicts a set 1100 of plots with simulated and measured data for the DFTUT using a hydrophone: (a) an axial beam profile with DOF and (b)-6 dB overall lateral beamwidth within the −6 dB DOF, in accordance with an exemplary embodiment of the present disclosure. The measured −6 dB DOF was approximately 14.5 mm as shown in FIG. 11(a). FIG. 11(b) shows measured −6 dB lateral beamwidths about 15 mm-30 mm in the −6 dB DOF, however, a dip was observed around 25 mm.

There are several possibilities that may explain this phenomenon including a difference in the delivered energies between the near field and far field and misalignment between two elements during the fabrication process, error of radius of each curvature, and the offset between two elements during pressed focusing. Because the most likely cause may be resulted from the difference in delivered energies due to the impedance and dimension of each element, a modified simulation was conducted by reducing the amplitude of the outer element signal to half of the inner element considering the amplitude of the dip in FIG. 11(a), also including geometrical errors of the prototype transducer such as dimension and the depth offset between two elements. However, it appeared that the most significant change came from the amplitude difference between the two frequencies. When the amplitude of low frequency component was decreased to half of the high frequency component in the input signal, the resulted curve was remarkably similar as shown in FIG. 11(a), (b). Although the location of the dip point is lower than measured data, the patterns were very similar. Thus, compared to same amplitude results in FIG. 5, it is clear that the output power of each element may affect the performance of DFTUT. It seems that this phenomenon might be caused by the electrical impedance mismatch due to dimension and driving frequency for each element. The alignment accuracy may be also critical during the fabrication process. These results showed that the extended −6 dB DOF could be obtained although it was not possible to maintain a constant lateral beamwidth throughout the DOF which likely was caused by the difference in the delivered energies as illustrated in FIG. 11(a). This unbalance in emitted power between the two elements may be minimized through more optimized transducer design carefully considering the impedance match, frequency, dimension of each element.

FIG. 12 depicts a plot with simulated and measured lateral beam pattern for the DFTUT: the simulated and the measured data at 20 mm and at 22 mm in depth (blue-dashed), in accordance with an embodiment of the present disclosure. FIG. 12 shows the measured lateral beam profiles at 20 mm and 22 mm depths with simulation data under the condition which is similar to FIG. 11. The measured and the simulated sidelobe level were −11.3 dB and −13.4 dB, respectively. The discrepancies between the two data sets may be attributed to the limited dynamic range of the 8-bit ADC card.

FIG. 13 depicts a set 1300 of plots of (a) Simulated temperature distribution and (b) thermal dose for the tested DFTUT by targeting a liver layer. The position of the transducer was on the bottom side. Note that the lateral axis scale of (b) is different from the (a).

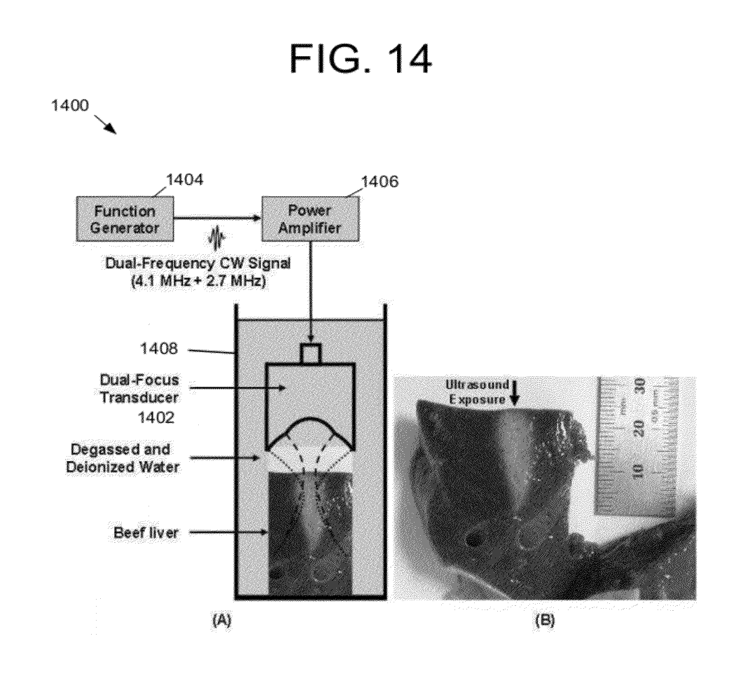

FIG. 14 depicts a schematic view of a DFTUT system and a cross-section of a piece of beef liver after HIFU sonication with DFTUT. The arrow indicates the HIFU exposure direction. FIG. 14(A) is a schematic diagram to of the system used to generate the extended DOF in the beef liver. FIG. 14(B) is a photograph of the ablated lesion in a beef liver. As shown in FIG. 14, a test on soft biological tissue lesion formation was conducted to verify the performance of the DFTUT system. The basic experimental arrangement is as shown in FIG. 14(A). DFTUT system includes a dual-focus transducer 1402 within a container 1408 of degassed and deionized water. Function generator 1404 is operable to generate a multi-frequency signal, e.g., as shown and described for FIG. 1. The multi-frequency signal was sent to the transducer 1402 by way of power amplifier 1406.

For the experiment, a fresh whole beef liver (shown dissected in FIG. 14(B), obtained from the slaughter house within 12 hours after excision from the animal, was sliced with 5 cm×5 cm×5 cm in size. The specimen was not degassed and placed in a degassed water bath. The target distance from the surface of the transducer was controlled to be approximately 10 mm with a sample holder.

The amplitude of the signal used to excite the transducer was increased to 140 Vₚₚ with a 55 dB power amplifier (A300, ENI Co., Santa Clara, Calif.) resulting in an estimated 6.1 MPa peak positive pressure. FIG. 14(B) shows the ablated tissue lesion of a beef liver with 140 Vₚₚ applied voltage for 30 seconds. The lesion size was approximately 20 mm in length and tapering width from about 8 mm. This tapering was likely due to the difference in the delivered energies between the two elements in the DFTUT. The coagulated region in the axial and lateral direction was wider than the results obtained by hydrophone measurements in FIG. 11.

Note that the coagulated lesion in FIG. 14 was formed in front of the geometrical focal depths of DFTUT and moved toward the surface of the tissue. This prefocal heating may be explained by the nonlinear distortion of the ultrasound wave in the tissue. Another reason may be the changed property of the ablated-tissue resulting in unusual attenuation coefficients during high temperature HIFU sonication such as thermal lens effect.

Several fabrication issues such as a depth offset and alignment errors during the assembly of the two elements can be solved or mitigation with the use of proper fixtures. Additionally, under similar driving conditions the ultrasound intensity along the elongated DOF produced by the DFTUT may be lower than a single focused transducer, so higher driving power may be required to obtain the same treatment effect. The bio-heat transfer simulation results show that the DFTUT can generate a temperature up to 83° C. under the current driving conditions within the extended DOF and the lesion formation test with a piece of beef liver shows a coagulated lesion of about 20 mm length and 8 mm width with a tapering shape, which may come from the relatively lower intensity of the outer element. Because the temperature distribution is not sufficient to estimate coagulated lesion size, thermal dose simulation was performed and the 240 min region was about 0.8 mm width and 12 mm length.

The simulated temperature profile shows the lesion of a maximal 5 mm width and 28 mm length with different temperature distribution. In the in vitro experiment, although the whole region with changed color due to HIFU sonication was about 8 mm width and 25 mm length, there was gradual color variation from the center to the outer zone. The more coagulated center lesion of white color was about 2 mm in width and 11 mm in length which corresponded to the thermal dose simulation results which showed a lesion of 1 mm width and 12 mm length at 240 min. This thermal lesion expansion may be explained by the thermal conduction due to high ambient temperature resulted from extended exposure time for about 30 seconds. The changed attenuation/absorption coefficients during HIFU sonication might increase the focal temperature (Meaney et al. 1998). The nonlinear wave distortion and cavitation might also play a role in the non-degassed target and thus resulted in an expanded lesion (Connor and Hynynen 2002; He et al. 2005). Because the classical bio-heat/thermal dose simulation and hydrophone measurement do not include these factors, significant discrepancy between the simulation and experiment may result (Connor and Hynynen 2002, Hallaj et al 2001).

Increasing the amplitude of the outer element excitation was necessary to obtain a more regular lesion shape to compensate the increased attenuation compensation for deep targets. Currently, 21 W electrical power input to the prototype transducer was almost close to its maximal capability to achieve lesion ablation. In the frequency mixed input signal, the amplitude of low frequency signal for the outer element should be approximately doubled to achieve proper intensity compensation based on simulation results. This compensation may be over the power limit of the current prototype transducer.

In the in vitro experiment, the target was mounted in the water and there was approximately a 10 mm water standoff. Among them, 6 mm was the distance from the center of the transducer to the front of the transducer's housing, and 4 mm was the distance from the front of the housing to the target. In this setup, the −6 dB pressure level measured by a hydrophone was considered expecting the proximal point of the lesion might be start few millimeters beneath from the surface of the target. However, the lesion was expanded to larger than that predicted by the −6 dB intensity contour and was formed close to the surface of the target, i.e., the proximal point of the lesion was about 1 mm below of the surface. One of the potential reasons causing this prefocal heating was the thermal conduction related to an extended sonication time. The changed attenuation/absorption coefficient and nonlinear distortion of the ultrasound wave in the ablated lesion during HIFU sonication may cause formation of the lesion in front of the geometrical focus, which may be similar to thermal lens effect.

Accordingly embodiments of the present disclosure can provide various benefits and advantages. The length of the extended DOF can be changed by controlling the relative geometric focus offset. The sidelobe level of DFTUT also can be varied by changing the physical dimensions of the multiple elements. Such transducers can be used to extend DOF resulting in a broad tissue lesion in the axial direction, which may be useful for treatment of the large tumors especially deep-seated tumors. The fabrication process for the DFTUT (or, a multi-element transducer) may be further improved to optimize the performance of such a device. Moreover, ultrasound transducers according to the present disclosure may increase the size of ablated tissues in the axial direction, thus decreasing the treatment time for a large volume of malignant tissues especially deep-seated targets.

One skilled in the art will appreciate that embodiments and/or portions of embodiments of the present disclosure can be implemented in/with computer-readable storage media (e.g., hardware, software, firmware, or any combinations of such), and can be distributed over one or more networks. Steps or operations (or portions of such) as described herein, including processing functions to derive, learn, or calculate formula and/or mathematical models utilized and/or produced by the embodiments of the present disclosure, can be processed by one or more suitable processors, e.g., processing units implementing suitable code/instructions in any suitable language (machine dependent on machine independent).

While certain embodiments and/or aspects have been described herein, it will be understood by one skilled in the art that other embodiments may be included within the scope of the present disclosure. For example, while various implementation parameters are described herein as being directed to treatment of tissue, embodiments of the present disclosure can be used for various other situations/applications, such as manufacturing applications, e.g., welding. Additionally, the piezoelectric materials and composite(s) utilized for embodiments described herein are merely representative and others may be used. Accordingly, the embodiments described herein, and as claimed in the attached claims, are to be considered in all respects as illustrative of the present disclosure and not restrictive.

What is claimed is:

1. An ultrasound system comprising:
a multi-element acoustic therapy transducer including a plurality of transducer elements, wherein each of the plurality of transducer elements is configured to produce a different focal zone along an axis.

2. The system of claim 1, further comprising a controller system configured and arranged to control the acoustic therapy transducer.

3. The system of claim 1, wherein the controller system comprises a single transmitter operable to generate a drive signal with multiple frequencies for driving the transducer.

4. The system of claim 1, wherein the controller system comprises a plurality of transmitters, each configured to generate a drive signal for a respective one of the plurality of transducer elements.

5. The system of claim 1, wherein the plurality of transducer elements are arranged in a concentric configuration.

6. The system of claim 1, wherein each of the plurality of transducer elements has a different radius of curvature.

7. The system of claim 1, wherein each of the plurality of transducer elements has a different thickness.

8. The system of claim 5, wherein the plurality of transducer elements includes a disc element and an annular element.

9. The system of claim 8, wherein the disc element is disposed within an inner aperture of the annular element.

10. The system of claim 6, wherein the plurality of transducer elements comprises three transducer elements.

11. The system of claim 6, wherein the plurality of transducer elements comprise an inner disc transducer element having a radius of curvature of about 19 mm and an outer annular transducer element having a radius of curvature of about 24 mm.

12. The system of claim 11, wherein the inner disc transducer element has a center frequency of about 2.7 MHz and the outer annular transducer element has a center frequency of about 2.7 MHz.

13. A method of aligning ultrasonic depths-of field, the method comprising:
providing a multi-element acoustic therapy transducer including a plurality of transducer elements, wherein each of the plurality of transducer elements is configured to produce a different focal zone along an axis; and
controlling the transducer to produce an ultrasonic output along the axis, wherein the ultrasonic depths-of field are aligned along the axis.

14. The method of claim 13, wherein the axis is in a targeted portion of tissue, for therapeutic ultrasound treatment.

15. The method of claim 13, wherein the controller system comprises a single transmitter operable to generate a drive signal with multiple frequencies for driving the transducer.

16. The method of claim 14, wherein the plurality of transducer elements are arranged in a concentric configuration.

17. The method of claim 13, wherein each of the plurality of transducer elements has a different radius of curvature.

18. The method of claim 13, wherein each of the plurality of transducer elements has a different thickness.

19. The method of claim 13, wherein the plurality of transducer elements includes a disc element and an annular element.

20. The method of claim 19, wherein the disc element is disposed within an inner aperture of the annular element.

21. The method of claim 14, further comprising effecting thrombolysis in the targeted portion of tissue.
